(19) United States
(12) Patent Application Publication
(10) Pub. No.: US 2001/0029264 A1
(43) Pub. Date: Oct. 11, 2001

(54) Novel methods and compositions for delivery of taxanes

(76) Inventor: Lisa L. McChesney-Harris, Vernon Hills, IL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/795,626

(22) Filed: Feb. 28, 2001

Related U.S. Application Data
(63) Continuation of application No. 09/265,649, filed on Mar. 10, 1999, now abandoned.
(60) Provisional application No. 60/077,459, filed on Mar. 10, 1998.

(51) Int. Cl. A61K 31/337; A61K 31/355

(52) U.S. Cl. 514/449; 514/458

(57) ABSTRACT

Novel methods and compositions of delivery of taxanes are disclosed. Particularly disclosed are compositions of taxol solubilized in Vitamin E TPGS whereby the delivery of taxol is improved.

### CROSS-REFERENCE TO A RELATED APPLICATION

This is a continuation of co-pending application Ser. No. 09/265,649, filed Mar. 10, 1999 which is incorporated herein by reference.

### BACKGROUND OF THE INVENTION

Taxanes make up an important class of cytotoxic agents which have been the subject of much interest and research directed to producing new and improved cancer-fighting therapies. A particularly promising taxane, paclitaxel, is a compound extracted from the bark of a western yew, Taxus brevifolia and known for its antineoplastic activity. It is described, for example, in The Merck Index, Eleventh Edition 1989, monograph 9049.

In 1977, paclitaxel was chosen for development as an antineoplastic agent because of its unique mechanism of action and good cytotoxic activity against IP implanted D16 melanoma and the human X-1 mammary tumor xenograft.

Paclitaxel is believed to function as a mitotic spindle poison and as a potent inhibitor of cell replication in vitro. Other mitotic spindle poisons (colchicine and podophyllotoxin) inhibit microtubule assembly. Paclitaxel employs a different mechanism of action since it appears to shift the equilibrium of polymerization/depolymerization toward polymer assembly and to stabilize microtubules against depolymerization under conditions which would cause rapid desegregation of microtubules. The interference with the polymerization/depolymerization cycle in cells appears to interfere with both the replication and migration of cells.

Paclitaxel has demonstrated good response rates in treating both ovarian and breast cancer patients who were not benefitting from vinca alkaloid or cisplatin therapy. It has also shown encouraging results in patients with other types of cancer including lung, melanoma, lymphoma, head, and neck. For further information, reference may be made to the U.S. National Cancer Institute's Clinical Brochure for Taxol, revised July 1991, and papers presented at the Second National Cancer Institute Workshop on Taxol and Taxus held in Alexandria, Va. U.S.A. on Sep. 23-24, 1992.

Despite these studies which affirm paclitaxel's importance as a tool in the fight against cancer, the chemical structure of paclitaxel creates obstacles for its efficient pharmaceutical administration. One such obstacle is that paclitaxel is water insoluble and tends to precipitate when placed in an aqueous solution. Some formulations of paclitaxel used for injection or IV infusion have been developed primarily utilizing CREMOPHOR® EL as the drug carrier to overcome the low water solubility problems of paclitaxel. Cremophor, however, is itself somewhat toxic, causing idiosyncratic histamine release and anaphylactoid like response. Thus, the use of this carrier is not a desirable solution to the problem of developing good formulations of taxanes.

Extensive efforts have been made to circumvent these problems inherent in the administration of paclitaxel. For example, in U.S. Pat. No. 4,942,184, Haugwitz et al. attempted to make paclitaxel more water soluble by altering its chemical structure. See also U.S. Pat. No. 4,960,790. This changing of the chemical structure of paclitaxel can potentially decrease the antitumor activity of the drugs, and does not address the problem of low stability and short shelf life.

There is a continuing need for taxane compositions and formulations which provide a more efficient means of administering taxanes without causing allergic reactions or other undesired side effects, and which have improved stability and longer shelf life.

### SUMMARY OF THE INVENTION

The subject invention pertains to novel methods and compositions for delivery of paclitaxel and other taxanes or their water insoluble derivatives. Specifically exemplified are compositions of paclitaxel solubilized in d-alpha-tocopheryl polyethylene glycol 1000 succinate and methods of making the same. The methods and compositions of the subject invention provide paclitaxel compositions which have improved stability and are suitable for oral or injectable administration.

One aspect of the subject invention pertains to methods of preparing taxane formulations comprising mixing Vitamin E TPGS with an organic solvent to form a carrier solution and contacting a taxane with said carrier solution, whereby said taxane is solubilized in said carrier solution and does not readily degrade. In a specific aspect of the subject invention, an acid is added to the organic solvent to reduce its pH before mixing with the Vitamin E TPGS, which acts to improve stability of the taxane. U.S. Pat. No. 5,733,888 teaches methods for stabilizing paclitaxel by reducing the pH of the carrier solution. The content of the '888 patent is hereby incorporated by this reference.

Another aspect of the subject invention pertains to novel compositions comprising a taxane, wherein said taxane is solubilized in Vitamin E TPGS micelles. A particular aspect of the subject invention is directed to compositions designed for oral administration or injectable administration.

### DETAILED DISCLOSURE OF THE INVENTION

The subject invention pertains to novel taxane compositions, and methods of making and administering the same. Specifically exemplified herein are compositions comprising taxanes, Vitamin E TPGS, and an organic solvent.

One aspect of the subject invention is directed to compositions comprising taxanes, Vitamin E TPGS, and ethanol. A specific embodiment of the subject invention utilizes paclitaxel as the taxane component.

The compositions of the subject invention may be formulated with or without further excipients. Examples of preferred compositions include, but are not limited to, the following:

a) solutions for drinking,

b) emulsions for drinking,

c) injection solutions, and

d) solutions contained in capsules.

The modes of administration include, but are not limited to, intramuscular, subcutaneous, intravenous, parenteral, and oral administration.

In a specific aspect, the pH of the carrier composition can be reduced to further improve the stability of the taxane contained in said composition. In some embodiments, this is accomplished by the addition of an acid. In a preferred embodiment, the acid is citric acid.

In other embodiments, the compositions of the subject invention can be contained within a gelatin capsule. Thickeners known in the art can be added to these compositions in order to make them more suitable for gelatin capsule administration. An example of such a thickener includes, but is not limited to, PEG 4600.

Another embodiment of the subject invention is directed to a method of preparing a taxane formulation comprising mixing Vitamin E TPGS with an organic solvent to form a carrier solution, and then contacting a taxane with said carrier solution, whereby said taxane is solubilized in said carrier solution and does not readily degrade. In a preferred embodiment, the desired amount of Vitamin E TPGS is warmed to approximately 40° C., and then stirred as the organic solvent is added. Organic solvents which can be used in the subject method include, but are not limited to, ethanol. The contacting of taxane with the carrier solution can be accomplished by adding the taxane to the carrier solution slowly, with continued stirring, at 40° C. The resulting composition can remain a fluid even after cooling to ambient temperature. In a preferred embodiment the resulting composition is maintained as an anhydrous solution. Upon administration of this anhydrous solution, it contacts aqueous bodily fluids whereby the solution emulsifies and forms taxane-containing micelles.

The percentages of the respective components of the subject compositions will vary depending on the type of administration contemplated. Compositions most suitable for oral administration will, in preferred embodiments, have a more solid or semi-solid consistency. Accordingly, compositions for oral administration will preferably comprise about 50% to about 75% Vitamin E-TPGS. In contrast, the most preferred embodiments of compositions ultimately intended for intravenous (IV) or parenteral administration, for example, will preferably comprise lesser amounts of Vitamin E-TPGS, ideally between about 25% to about 60% Vitamin E-TPGS. Compositions ultimately intended for IV or parenteral administration can also comprise an organic solvent, preferably in an amount of about 40% to about 75%.

The present invention may be understood more readily by reference to the following descriptions of preferred embodiments and examples of the invention. Other embodiments within the scope of the invention will be readily apparent to those of skill in the art in view of the teachings herein.

### EXAMPLE 1

### Surfactant/Solvent Combinations Comprising a Taxane Component

4.040 g of Vitamin E-TPGS (Eastman Chemical Co., Kingsport, Tenn.) was chipped in and weighed into a 20 ml scintillation vial. 0.514 g of dimethylisosorbide (“DMI”; ARLASOLVE® DMI, ICI Surfactants; Wilmington, Del.) was added to the vial followed by heating to melt the Vitamin E-TPGS/DMI mixture. Upon liquification of the Vitamin E-TPGS/DMI mixture, 0.248 g of paclitaxel was added with stirring. Unexpectedly, the taxane rapidly dispersed and the particles solubilized quickly. The solution began to clarify and dissolution progressed. The solution composition was adjusted to determine how much paclitaxel it could efficiently solubilize. Accordingly, additional amounts of Vitamin E-TPGS/DMI, and paclitaxel were added to the mixture with stirring. The majority of the bulk paclitaxel dissolved in the warmed, stirred matrix. Final concentrations were as follows: 7.065 g Vitamin E-TPGS, 2.014 g dimethylisosorbide, and 0.664 g paclitaxel.

To test the potential for drug precipitation, a small quantity of the warmed mixture was transferred via a plastic transfer pipette to a 20 ml scintillation vial containing cold water. The drop of formulation immediately congealed (solidified) on the surface of the water and formed a clear, gelatinous-like mass. Upon agitation, the mass dissolved and a large number of bubbles were visible on the surface of the solution. However, there was no visible precipitation of any drug particles. Approximately 24 hours later, there still appeared to be many bubbles at the surface and the presence of a very thin, opaque film could be seen on the bottom of the vial. This sedimentation was possibly paclitaxel that had partitioned out of the micelles.

The remaining original Vitamin E-TPGS, DMI, and paclitaxel solution mixture was also allowed to cool to room temperature. The mixture congealed to form a semi-solid, light amber yellow mass. No apparent phase separation could be seen to indicate possible incompatibility. No crystals appeared to be present in the matrix.

A small quantity of congealed formulation was transferred to a separate 20 ml scintillation vial and 3 to 4 ml of warm water were added. The sample was agitated gently and visually monitored. The semi-solid mass quickly hydrated and became translucent with no evidence of precipitation of active components either within the gelatinous matrix or in the aqueous phase.

The experiment as described above was repeated, and the results were reproducible.

### EXAMPLE 2

### Vitamin E-TPGS Formulation Optimization for Solubilizing Paclitaxel

Various concentrations of CREMOPHOR® EL/citric acid blend, DMI, Vitamin E-TPGS, and paclitaxel were mixed together to determine preferred formulations for solubilizing paclitaxel. See Table 1. Each formulation was prepared in a 20 ml scintillation vial. The vial was initially tared. CREMOPHOR® EL/citric acid blend (10 g CREMOPHOR® EL, 0.020 g citric acid) was weighed into each of eight (8) tared vials. The weight was recorded. Each vial was subsequently tared and the appropriate weight of dimethylisosorbide (DMI) was added. An additional five (5) vials were also prepared without containing the CREMOPHOR® EL/citric acid blend. These are identified as Samples 9-13 of Table 1. All vials were individually tared and the appropriate weight of Vitamin E-TPGS was added. At this point, all vials were placed in a 40° C. incubator to liquefy the Vitamin E-TPGS within each sample. After the solutions had liquefied, two samples were removed at a time and placed on a stir/hotplate. One-half inch egg shaped stir bars were added to each scintillation vial.

Small weighing canoes were tared on the balance and paclitaxel was transferred until the desired weight was achieved. Large clumps were broken apart easily with a stainless steel spatula. The paclitaxel was slowly added to the warmed, stirring mixtures of Samples 1 and 2. Despite the presence of CREMOPHOR® EL, the bulk drug dissipated quickly and the solutions clarified relatively quickly. The last few particles took a little longer to dissolve, but eventually did, and the taxane retained its solubility even in the presence of 10% CREMOPHOR® EL. This same procedure was repeated for each of the samples.

Formulations were prepared as above wherein the DMI was substituted by methoxylated PEG 350. See Table 2.

TABLE 1

| | CREMOPHOR ® | | | | | | | |
|---|---|---|---|---|---|---|---|---|
| | EL, | | | | | | | |
| | Citric Acid Blend | | DMI | | Vitamin E-TPGS | | Paclitaxel | |
| Sample | Desired | Actual | Desired | Actual | Desired | Actual | Desired | Actual |
| ID | Amount | Amount | Amount | Amount | Amount | Amount | Amount | Amount |
| 1 | 0.50 g | 0.498 | 1.00 g | 1.013 | 3.25 g | 3.269 | 0.25 g | 0.251 |
| 2 | 0.50 g | 0.498 | 1.00 g | 1.006 | 3.20 g | 3.206 | 0.30 g | 0.304 |
| 3 | 0.50 g | 0.507 | 1.00 g | 1.019 | 3.15 g | 3.158 | 0.35 g | 0.350 |
| 4 | 0.50 g | 0.499 | 1.25 g | 1.252 | 2.75 g | 2.803 | 0.50 g | 0.500 |
| 5 | 0.50 g | 0.551 | 1.25 g | 1.261 | 3.00 g | 3.011 | 0.25 g | 0.251 |
| 6 | 0.50 g | 0.495 | 1.25 g | 1.267 | 2.95 g | 2.943 | 0.30 g | 0.304 |
| 7 | 0.50 g | 0.522 | 1.25 g | 1.266 | 2.90 g | 2.909 | 0.35 g | 0.353 |
| 8 | 0.50 g | 0.524 | 1.50 g | 1.500 | 2.50 g | 2.510 | 0.50 g | 0.506 |
| 9 | na | | 1.00 g | 1.013 | 3.75 g | 3.770 | 0.25 g | † |
| 10 | na | | 1.00 g | 1.026 | 3.70 g | 3.737 | 0.30 g | † |
| 11 | na | | 1.00 g | 1.005 | 3.65 g | 3.645 | 0.35 g | 0.354 |
| 12 | na | | 1.25 g | 1.246 | 3.25 g | 3.243 | 0.50 g | 0.506 |
| 13 | na | | 1.50 g | 1.518 | 3.00 g | 3.004 | 0.50 g | † |

†These solutions were not prepared, i.e., no drug added due to success of Samples 11 and 12.

TABLE 2

| | CREMOPHOR ® | | | | | | | |
|---|---|---|---|---|---|---|---|---|
| | EL, Citric Acid | | Methoxylated PEG | | | | | |
| | Blend | | 350 | | Vitamin E-TPGS | | Paclitaxel | |
| Sample | Desired | Actual | Desired | Actual | Desired | Actual | Desired | Actual |
| ID | Amount | Amount | Amount | Amount | Amount | Amount | Amount | Amount |
| 14 | 0.50 g | 0.510 g | 1.25 g | 1.262 g | 2.95 g | 2.953 g | 0.30 g | 0.303 g |
| 15 | 0.50 g | † | 1.25 g | † | 2.75 g | † | 0.50 g | † |
| 16 | na | | 1.25 g | 1.252 g | 3.45 g | 3.478 g | 0.30 g | 0.302 g |
| 17 | na | | 1.25 g | 1.254 g | 3.25 g | 3.260 g | 0.50 g | 0.504 g |

†not prepared.

The same type of dissolution was observed for methoxy PEG 350 as for DMI; it initially became an opaque dispersion which clarified over time.

All samples were surprisingly able to solubilize the specified amount of paclitaxel added. The addition of 10% CREMOPHOR® EL seemed to retard the dissolution process, but it did not cause any problems preventing final dissolution of all of the paclitaxel.

All of the samples were allowed to cool to room temperature. Upon cooling, all of the solutions appeared to remain intact, i.e., no phase separation or precipitation of paclitaxel was visible. The samples containing 10% CREMOPHOR® EL and 25% DMI (Samples 5-8) appeared non-homogeneous, with Vitamin E-TPGS precipitating out within a light amber solution making two phases apparent. Samples 1-4 and 11, 12, 14, and 16 were opaque semi-solids demonstrating no apparent precipitation of active ingredient.

The ability of the methoxy PEG compounds to solubilize paclitaxel alone was also tested. Formulations were prepared according to Table 3. Methoxy PEG 350 quickly dissolved both levels of paclitaxel. Methoxy PEG 550 also eventually dissolved the paclitaxel, but it was a slower process.

TABLE 3

| Desired Amount | Actual Amount | Desired Amount | Actual Amount |
|---|---|---|---|
| Methoxylated PEG 350 | | Paclitaxel | |
| 0.450 g | 0.456 g | 0.050 g | 0.051 g |
| 0.900 g | 0.901 g | 0.100 g | 0.107 g |
| Methoxylated PEG 550 | | Paclitaxel | |
| 0.450 g | 0.462 g | 0.050 g | 0.049 g |

### EXAMPLE 3

### Micellar Solubilization

All 20 ml scintillation vials containing the samples described in Examples 1 and 2 were placed in a 40° C. incubator. Plastic transfer pipettes were also placed in the 40° C. incubator to minimize congealing during transfer. Each sample was removed and a warmed pipette was utilized to transfer aliquots into three (3) vials and into a number 1 size hard gelatin capsule. See Table 4.

TABLE 4

| | Amount | Amount | Amount | Amount of | Amount |
|---|---|---|---|---|---|
| | Weighed | Weighed | Weighed | Water | Weighed into |
| Sample | into | into | into | Added | Gelatin |
| ID | Vial 1 | Vial 2 | Vial 3 | to Vial 3 | Capsule |
| 1 | 1.054 g | 1.042 g | 1.006 g | 0.048 g | 0.400 g |
| 2 | 1.085 g | 1.012 g | 0.999 g | 0.042 g | 0.442 g |
| 3 | 1.040 g | 1.008 g | 0.996 g | 0.041 g | 0.447 g |
| 4 | 1.020 g | 1.036 g | 1.008 g | 0.041 g | 0.430 g |
| 5 | 1.042 g | 0.997 g | 0.996 g | 0.048 g | 0.431 g |
| 6 | 1.009 g | 1.047 g | 0.996 g | 0.054 g | 0.436 g |
| 7 | 1.017 g | 0.994 g | 1.000 g | 0.045 g | 0.438 g |
| 8 | 1.087 g | 1.013 g | 1.004 g | 0.045 g | 0.412 g |
| 9 | N/A | N/A | N/A | N/A | N/A |
| 10 | N/A | N/A | N/A | N/A | N/A |
| 11 | 1.003 g | 0.996 g | 1.004 g | 0.046 g | 0.446 g |
| 12 | 1.035 g | 1.013 g | 1.014 g | 0.047 g | 0.441 g |
| 13 | N/A | N/A | N/A | N/A | N/A |
| 14 | 1.020 g | 1.049 g | 1.022 g | 0.043 g | 0.434 g |
| 15 | N/A | N/A | N/A | N/A | N/A |
| 16 | 1.014 g | 1.013 g | 0.995 g | 0.049 g | 0.463 g |
| 17 | 0.996 g | 1.021 g | 1.005 g | 0.067 g | 0.470 g |

N/A = did not prepare designated formula

One vial was stored at ambient room temperature, one vial stored at 40° C., one vial was filled with approximately 1 g of sample and approximately 0.05 g of water (vortexed and placed at 40° C.), and one number 1 size capsule was filled (approximately 15 drops) and capped (stored at ambient room temperature in a capped 1 dram, 14.5×45 mm, opticlear vial).

(Sample ID #1—10% CREMOPHOR® EL, 20% DMI, 5% paclitaxel, 65% Vitamin E-TPGS)

| | Sample Description | Observations |
|---|---|---|
| Rep 1 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 2 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 3 | Vial 1 RT | some phase separation visible |
| | Vial 2 40° C. | clear, pale yellow liquid |
| | Vial 3 + water | clear, pale yellow liquid |
| | Capsule RT | opaque, waxy solid, light yellow |

(Sample ID #2—10% CREMOPHOR® EL, 20% DMI, 6% paclitaxel, 64% Vitamin E-TPGS)

| | Sample Description | Observations |
|---|---|---|
| Rep 1 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 2 | Vial 1 RT | opaque, waxy solid, light yellow |
| | Vial 2 40° C. | clear, a few strands of shiny particulate |
| | Vial 3 + water | pale yellow liquid with a few strands of |
| | | shiny ppt (40° C.) |
| | Capsule RT | opaque, waxy solid, light yellow |
| Rep 3 | Vial 1 RT | opaque, waxy solid, light yellow |
| | Vial 2 40° C. | clear, few strands of shiny ppt |
| | Vial 3 + water | clear, pale yellow liquid with few strands of |
| | | ppt |
| | Capsule RT | opaque, waxy solid, light yellow |

(Sample ID #3—10% CREMOPHOR® EL, 20% DMI, 7% paclitaxel, 63% Vitamin E-TPGS)

| | Sample Description | Observations |
|---|---|---|
| Rep 1 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 2 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |

(Sample ID #4—10% CREMOPHOR® EL, 20% DMI, 10% paclitaxel, 60% Vitamin E-TPGS)

| | Sample Description | Observations |
|---|---|---|
| Rep 1 | Vial 1 RT | multiple phases visible, striation |
| | Vial 2 40° C. | clear, pale yellow liquid |
| | Vial 3 + water | clear, pale yellow liquid (40° C.) |
| | Capsule RT | apparent phase separation |
| Rep 2 | Vial 1 RT | some striation at bottom, otherwise waxy |
| | | mass |
| | Vial 2 40° C. | clear, pale yellow liquid |
| | Vial 3 + water | clear, pale yellow liquid (40° C.) |
| | Capsule RT | apparent multiple phases |

(Sample ID #5—10% CREMOPHOR® EL, 25% DMI, 5% paclitaxel, 60% Vitamin E-TPGS)

| | Sample Description | Observations |
|---|---|---|
| Rep 1 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 2 | Vial 1 RT | opaque, waxy solid, light yellow |
| | Vial 2 40° C. | light yellow liquid with some shiny |
| | | crystalline ppt |
| | Vial 3 + water | light yellow liquid with some shiny, |
| | | crystalline ppt (40° C.) |
| | Capsule RT | opaque, waxy solid, light yellow |
| Rep 3 | Vial 1 RT | definite phase separation, light yellow, clear |
| | | and solid off-white wax |
| | Vial 2 40° C. | light yellow liquid with some shiny ppt |
| | Vial 3 + water | light yellow liquid with some shiny ppt |
| | Capsule RT | multiple phases visible, intermingled |

(Sample ID #6—10% CREMOPHOR® EL, 25% DMI, 6% paclitaxel, 59% Vitamin E-TPGS)

| | Sample Description | Observations |
|---|---|---|
| Rep 1 | Vial 1 RT | apparent phase separation, striated |
| | Vial 2 40° C. | clear, pale yellow liquid |
| | Vial 3 + water | clear, pale yellow liquid (40° C.) |
| | Capsule RT | apparent phase separation |
| Rep 2 | Vial 1 RT | opaque, waxy solid, off-white |
| | Vial 2 40° C. | light yellow liquid with some shiny |
| | | crystalline ppt (40° C.) |
| | Vial 3 + water | light yellow liquid with some shiny, |
| | | crystalline ppt (40° C.) |
| | Capsule RT | opaque, waxy solid, off-white |
| Rep 3 | Vial 1 RT | definite phase separation, light yellow, clear |
| | | and solid off-white wax |
| | Vial 2 40° C. | light yellow liquid with some shiny ppt |
| | Vial 3 + water | light yellow liquid with some shiny ppt |
| | Capsule RT | multiple phases visible, intermingled |

(Sample ID #7—10% CREMOPHOR® EL, 25% DMI, 7% paclitaxel, 58% Vitamin E-TPGS)

| | Sample Description | Observations |
|---|---|---|
| Rep 1 | Vial 1 RT | apparent phase separation, striated |
| | Vial 2 40° C. | clear, pale yellow liquid |
| | Vial 3 + water | clear, pale yellow liquid (40° C.) |
| | Capsule RT | apparent phase separation |
| Rep 2 | Vial 1 RT | congealed with striations |
| | Vial 2 40° C. | light yellow liquid with some shiny |
| | | crystalline ppt |
| | Vial 3 + water | light yellow liquid with some shiny, |
| | | crystalline ppt (40° C.) |
| | Capsule RT | congealed with striations |
| Rep 3 | Vial 1 RT | multiphase appearance |
| | Vial 2 40° C. | light yellow, clear liquid with shiny ppt |
| | Vial 3 + water | light yellow, clear liquid with shiny ppt |
| | Capsule RT | multiphase appearance |

(Sample ID #8—10% CREMOPHOR® EL, 25% DMI, 10% paclitaxel, 55% Vitamin E-TPGS)

| | Sample Description | Observations |
|---|---|---|
| Rep 1 | Vial 1 RT | apparent phase separation, striated |
| | Vial 2 40° C. | clear, pale yellow liquid |
| | Vial 3 + water | clear, pale yellow liquid (40° C.) |
| | Capsule RT | completely clear solution |
| Rep 2 | Vial 1 RT | liquid and solid phases visible, liquid on top |
| | Vial 2 40° C. | light yellow liquid with some shiny |
| | | crystalline ppt |
| | Vial 3 + water | light yellow liquid with some shiny, |
| | | crystalline ppt (40° C.) |
| | Capsule RT | liquid and solid phases visible |
| Rep 3 | Vial 1 RT | multiphase system in appearance |
| | Vial 2 40° C. | light yellow, clear liquid with shiny ppt |
| | Vial 3 + water | light yellow, clear liquid with shiny ppt |
| | Capsule RT | clear, pale yellow liquid |

(Sample ID #11—20% DMI, 7% paclitaxel, 73% Vitamin E-TPGS)

| | Sample Description | Observations |
|---|---|---|
| Rep 1 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 2 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |

(Sample ID #12—25% DMI, 10% paclitaxel, 70% Vitamin E-TPGS)

| | Sample Description | Observations |
|---|---|---|
| Rep 1 | Vial 1 RT | apparent phase separation, striated |
| | Vial 2 40° C. | clear, pale yellow liquid |
| | Vial 3 + water | clear, pale yellow liquid (40° C.) |
| | Capsule RT | apparent phase separation |
| Rep 2 | Vial 1 RT | semi-solid with striations |
| | Vial 2 40° C. | clear, pale yellow liquid |
| | Vial 3 + water | clear, pale yellow liquid (40° C.) |
| | Capsule RT | semi-solid with striations |

(Sample ID #14—10% CREMOPHOR® EL, 25% MPEG, 6% paclitaxel, 59% Vitamin E-TPGS)

| | Sample Description | Observations |
|---|---|---|
| Rep 1 | Vial 1 RT | opaque, off-white waxy solid |
| | Vial 2 40° C. | clear, pale yellow liquid |
| | Vial 3 + water | clear, pale yellow liquid (40° C.) |
| | Capsule RT | opaque, off-white waxy solid |
| Rep 2 | Vial 1 RT | opaque, off-white waxy solid |
| | Vial 2 40° C. | clear, pale yellow liquid |
| | Vial 3 + water | clear, pale yellow liquid (40° C.) |
| | Capsule RT | opaque, off-white waxy solid |

To test the micellar solution capability of the above samples, Samples 4, 8, and 12 were added to water. Formula composition 4=10% CREMOPHOR® EL, 25% DMI, 10% paclitaxel, and 55% Vitamin E-TPGS. Formula composition 8=10% CREMOPHOR® EL, 30% DMI, 10% paclitaxel, and 50% Vitamin E-TPGS. Formula composition 12=25% DMI, 10% paclitaxel, and 65% Vitamin E-TPGS. Five grams of water were added into 20 ml scintillation vials. 3 separate vials were prepared, each containing approximately 5 ml of water, and to each approximately 5 drops of one of formulations 4, 8, and 12 were added. Each vial was agitated to see if any precipitation occurred. Formulations 4 and 12 yielded clear solutions upon agitation. Formulation 8 immediately yielded a slightly turbid solution. This visual condition suggests that the portions of DMI and CREMOPHOR® EL may be disrupting the assembly of the micelles. It is believed that the micelles are formed as Vitamin E-TPGS hydrates to form a cubic phase structure. The drug is encapsulated and eventually released into solution as this process progresses. One explanation for this is that the quantities of DMI/CREMOPHOR® EL present cause the immediate dissolution of Vitamin E-TPGS, inhibiting the excipient from forming a transient cubic phase.

Both Formulations 4 and 12 became clear with many bubbles at the surface. The drops of formulation became gelatinous (process of hydrating) and then slowly dissolved. The final solutions were clear. After twelve hours, the aqueous solution containing 5 drops of Formulation 4 produced a thin film of precipitated material on the bottom of the vial. Formulation 12 remained clear with no evidence of precipitated material after twelve hours.

### EXAMPLE 4

### Taxane-Containing Gel Formulations

1% (0.05 g) paclitaxel was dissolved in 4.75 g of dimethylisosorbide. 0.20 g of KLUCEL® HF was sprinkled over the surface while the solution was rapidly stirred. This was carried out at room temperature using 20 ml scintillation vials containing a one-half inch egg-shaped stir bar placed on a Corning stir plate. The solution was clear and fluid upon completion. After sitting for approximately 30 minutes, the solution gelled.

### EXAMPLE 5

### Semi-Solid Formulations for Oral Administration

A container of Vitamin E-TPGS was placed in a 40° C. incubator, to liquefy the excipient. Plastic transfer pipettes were also warmed in the incubator to transfer the warmed excipient. Each formulation was again prepared in a 20 ml scintillation vial containing a one-half inch egg-shaped stir bar. The vial and stir bar were initially tared. Warmed Vitamin E-TPGS was dispensed to each tared vial using a warmed plastic transfer pipette. Each vial was subsequently tared and the desired amount of DMI was added using a plastic transfer pipette. Each vial was subsequently tared and the desired amount of PEG 4600 flake was added to two of the three vials. See Table 5 (Samples 19 and 20). Two scintillation vials (Samples 18 and 19) were placed on a stir/hotplate (the formulations containing PEG 4600 were given slightly increased heat as compared to Sample 18 to aid in melting the thickener). Small weighing canoes were tared on a balance and paclitaxel was transferred until the desired weight was achieved. Large clumps were broken apart easily with the stainless steel spatula. The paclitaxel was slowly added to the warmed, stirring excipients of the Samples 18 and 19.

TABLE 5

| | PEG 4600 Flake | | DMI | | Vitamin E-TPGS | | Taxanes | |
|---|---|---|---|---|---|---|---|---|
| Sample | Desired | Actual | Desired | Actual | Desired | Actual | Desired | Actual |
| ID | Amount | Amount | Amount | Amount | Amount | Amount | Amount | Amount |
| 18 | na | — | 1.00 g | 0.993 g | 3.50 g | 3.505 g | 0.50 g | 0.505 g |
| 19 | 0.25 g | 0.259 g | 1.00 g | 0.996 g | 3.25 g | 3.254 g | 0.50 g | 0.502 g |
| 20 | 0.25 g | 0.259 g | 1.25 g | 1.252 g | 3.00 g | 3.015 g | 0.50 g | 0.501 g |

The bulk drug again dissipated quickly and the solutions clarified rapidly.

The same procedure outlined above was used to prepare the final formulation of Sample 20.

Formulations were allowed to cool and were stored at ambient room temperature. To further characterize Formulations 18-20 they were subjected to the same procedures outlined in Example 3. Formulations 18-20 were warmed to liquefy and allow easier transfer. Transfer pipettes were also warmed. Three vials were prepared for each of Formulations 18-20, a, total of nine vials, each vial containing 1 g of formula. In addition, approximately 15 drops of each formulation were filled into a number 1 hard gel capsule. To the third vial of each formula, one drop of water was added and the sample vortexed. See Table 6.

TABLE 6

| | Amount | Amount | Amount | Amount of | Amount |
|---|---|---|---|---|---|
| | Weighed | Weighed | Weighed | Water | Weighed into |
| Sample | into | into | into | Added | Gelatin |
| ID | Vial 1 | Vial 2 | Vial 3 | to Vial 3 | Capsule |
| 18 | 1.023 g | 1.057 g | 1.007 g | 0.058 g | 0.445 g |
| 19 | 1.013 g | 1.038 g | 1.000 g | 0.055 g | 0.433 g |
| 20 | 0.999 g | 1.016 g | 1.002 g | 0.052 g | 0.418 g |

Five drops of each warmed solution were added to approximately 5 ml of water. At this time, the behavior of Samples 16 and 17 were also tested by introduction to approximately 5 ml of water.

Formulation 16—Approximately 5 g (˜5 ml) of water were transferred to a 20 ml scintillation vial. 5 drops of formulation were added (˜150 mg) and the mixture agitated slightly and continuously.

The drops of formulation congealed immediately upon striking the surface of the water (˜20° C.). The mass sank to the bottom and at first seemed to be just a precipitated globule of product. However, after 1 to 2 minutes of mild agitation, a translucent, crystal clear erosion layer could be seen around the entire aqueous exposed surface of the mass. Eventually, the entire mass dissolved as the erosion layer receded into the interior of the mass. The translucent, clear erosion layer appeared to be about 2 mm in thickness. The center remained opaque and solid until the erosion layer consumed the entire mass. The resulting solution was clear with a significant number of surfactant bubbles at the surface.

Formulation 17—This formulation behaved exactly the same as Formulation 16. The only difference between the two was drug loading, 6% for 16, 10% for 17.

Formulation 18 (20% DMI, 10% paclitaxel, 70% TPGS)—Five drops into 5 ml water; slightly agitated solution of formulation congealed upon striking the cool water (˜20° C.) and slowly dissolved into the aqueous solution. The mass (entire mass) quickly became translucent and gelatinous in appearance. Eventually all of the mass dissolved, yielding an aqueous solution with no apparent precipitated drug.

Formulation 19 (5% PEG 4600, 20% DMI, 10% paclitaxel, 65% TPGS)—Five drops into 5 ml water; slightly agitated. This formulation behaved essentially the same as Formulation 18. However, upon striking the cool water (˜20° C.), the mass seemed to elongate and disperse in the aqueous fraction. Striations were observed within the mass, which rapidly became translucent. The mass appeared to be like a bunch of spaghetti noodles structured together and wrapped around each other. The mass spread out along the bottom of the vial and appeared much more fluid than when PEG 4600 is absent in the formulation. Eventually, the mass dissolved, leading again to a clear solution with many bubbles at the surface.

Formulation 20 (5% PEG 4600, 25% DMI, 10% paclitaxel, 60% TPGS)—This formulation behaved just like Formulation 19.

(Sample ID #16—25% MPEG, 6% paclitaxel, 69% Vitamin E-TPGS)

| | Sample Description | Observations |
|---|---|---|
| Rep 1 | Vial 1 RT | opaque, off-white waxy solid |
| | Vial 2 40° C. | clear, pale yellow liquid |
| | Vial 3 + water | clear, pale yellow liquid (40° C.) |
| | Capsule RT | opaque, off-white waxy solid |
| Rep 2 | Vial 1 RT | opaque, off-white waxy solid |
| | Vial 2 40° C. | clear, pale yellow liquid |
| | Vial 3 + water | clear, pale yellow liquid (40° C.) |
| | Capsule RT | opaque, off-white waxy solid |

(Sample ID #17—25% MPEG, 10% paclitaxel, 65% Vitamin E-TPGS)

| | Sample Description | Observations |
|---|---|---|
| Rep 1 | Vial 1 RT | opaque, off-white waxy solid |
| | Vial 2 40° C. | clear, pale yellow liquid |
| | Vial 3 + water | clear, pale yellow liquid (40° C.) |
| | Capsule RT | opaque, off-white waxy solid |
| Rep 2 | Vial 1 RT | opaque, off-white waxy solid |
| | Vial 2 40° C. | clear, pale yellow liquid |
| | Vial 3 + water | clear, pale yellow liquid (40° C.) |
| | Capsule RT | opaque, off-white waxy solid |

(Sample ID #18—20% DMI, 10% paclitaxel, 70% Vitamin E-TPGS)

| | Sample Description | Observations |
|---|---|---|
| Rep 1 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |
| Rep 2 | Vial 1 RT | opaque, waxy solid, light yellow |
| | Vial 2 40° C. | clear, pale yellow liquid |
| | Vial 3 + water | clear, pale yellow liquid (40° C.) |
| | Capsule RT | opaque, waxy solid, light yellow |

(Sample ID #19—20% DMI, 5% PEG 4600, 10% paclitaxel, 65% Vitamin E-TPGS)

| | Sample Description | Observations |
|---|---|---|
| Rep 1 | Vial 1 RT | opaque, off-white waxy solid |
| | Vial 2 40° C. | material ppt on bottom, probably PEG 4600 |
| | Vial 3 + water | clear, pale yellow liquid (40° C.) |
| | Capsule RT | opaque, off-white waxy solid |
| Rep 2 | Vial 1 RT | opaque, off-white waxy solid |
| | Vial 2 40° C. | material ppt on bottom, probably PEG 4600 |
| | Vial 3 + water | clear, pale yellow liquid (40° C.) |
| | Capsule RT | opaque, off-white waxy solid |
| Rep 3 | Vial 1 RT | opaque, off-white waxy solid somewhat rigid |
| | Vial 2 40° C. | definite phase separation |
| | Vial 3 + water | clear, yellow liquid with a lot of ppt |
| | Capsule RT | opaque, off-white waxy solid |

(Sample ID #20—25% DMI, 5% PEG 4600, 10% paclitaxel, 60% Vitamin E-TPGS)

| | Sample Description | Observations |
|---|---|---|
| Rep 1 | Vial 1 RT | opaque, off-white waxy solid |
| | Vial 2 40° C. | material ppt on bottom, probably PEG 4600 |
| | Vial 3 + water | clear, pale yellow liquid (40° C.) |
| | Capsule RT | opaque, off-white waxy solid |
| Rep 2 | Vial 1 RT | opaque, off-white waxy solid |
| | Vial 2 40° C. | material ppt on bottom, probably PEG 4600 |
| | Vial 3 + water | clear, pale yellow liquid (40° C.) |
| | Capsule RT | opaque, off-white waxy solid |
| Rep 3 | Vial 1 RT | opaque, off-white waxy solid granular in |
| | | appearance |
| | Vial 2 40° C. | definite phase separation |
| | Vial 3 + water | clear, pale yellow liquid |
| | Capsule RT | opaque, off-white waxy solid |

Formulations 19 and 20, containing 5% PEG 4600, exhibited two main characteristics: (1) precipitation was visible in those formulations that contained 5% PEG 4600 and were kept at 40° C.; and (2) no precipitation was visible in those solutions containing 5% PEG 4600, kept at 40° C., and which contained 5% added water. These observations indicated that added water actually helps maintain the PEG 4600 in solution. The desired thickening effect of the PEG 4600 thus may require water to be present in the matrix to maintain a homogeneous mass.

### EXAMPLE 6

### Vitamin E-TPGS Taxane Formulations using PEG 300 as a Co-Solvent

Due to the success of the methoxypolyethylene glycol 350 formulations, PEG 300 was pursued as another possible candidate for a co-solvent in the Vitamin E-TPGS formulations. PEG 400 has shown some success in solubilizing taxanes. However, PEG 300 would be particularly desirable as a co-solvent because it is already approved for oral use in prescription drugs.

Vitamin E-TPGS was warmed in a 40° C. oven and once it was fluid, the desired quantity was transferred using a plastic transfer pipette into a 20 ml scintillation vial containing a stir bar. To the liquidified Vitamin E-TPGS the desired amount of PEG 300 was added to the vial. The PEG 300/Vitamin E-TPGS mixture was stirred while being warmed on the stir/hotplate. Paclitaxel was added slowly, with stirring, to the warmed PEG 300/Vitamin E-TPGS mixture. The mixture was stirred while warm until all of the paclitaxel dissolved. The formulation was then distributed into three (3) vials and one (1) hard gelatin capsule, as described above. One vial was stored at room temperature along with the capsule; the other two vials were placed in a 40° C. oven.

(Sample ID No. 21—25% PEG 300, 65% Vitamin E TPGS, 10% paclitaxel)

| | Sample Description | Observations |
|---|---|---|
| Rep 1 | Vial 1 RT | opaque, waxy solid, light yellow |
| | Vial 2 40° C. | clear, light yellow solution |
| | Vial 3 + Water | clear, light yellow solution (40° C.) |
| | Capsule RT | opaque, waxy solid, light yellow |

The samples were clear, amber yellow solutions while being warmed. The vial which was allowed to cool to room temperature became a yellow, waxy solid mass. While still warm, about 5 drops of the warmed formulation was transferred and added to about 5 ml of water and agitated. The mass initially solidified, but subsequently slowly dissolved into the aqueous layer. Eventually all of the mass dissipated into the water, and bubbles were apparent on the surface of the water. All of the paclitaxel was dissolved without any visually apparent precipitate.

### EXAMPLE 7

### Preparation of a Vitamin E-TPGS Formulation Containing Paclitaxel Suitable for Parenteral Delivery

The current commercially available formulation of paclitaxel is provided as a 50/50 mixture CREMOPHOR® EL/ethanol. Some of the formulations which have been described in the examples above are not optimally suited for parenteral delivery due to their solid or semi-solid consistency. This physical state does not lend itself well to sterilization technology. Accordingly, it was desirable to develop a liquid formulation particularly suitable for sterile filtration.

1.016 g of ethyl alcohol was transferred to a 20 ml scintillation vial containing a stir bar. 1.023 g of Vitamin E-TPGS was added to the vial. The solution was warmed slightly to facilitate the dissolution of the Vitamin E-TPGS. Upon dissolution of Vitamin E-TPGS, 0.205 g of paclitaxel was added with stirring. The paclitaxel dissolved quickly. To determine if the high level of co-solvent disturbs the ability of Vitamin E-TPGS to capture paclitaxel into micelles, approximately 6-8 drops of formulation were transferred to about 5 ml of water. Upon addition of the formulation to the water, the mixture was slightly agitated, which yielded a turbid solution. This turbidity is indicative of microprecipitation, suggesting that the cubic phase of Vitamin E-TPGS had been disrupted, thereby allowing the paclitaxel to precipitate when added to water. However, the turbid solution was not discarded, but was allowed to sit for a period of time. Surprisingly, the microparticulate dissipated and the solution appeared very clear with some bubbles on the surface of the water. This demonstrated that it is not necessary that the active component be captured and internalized during dissolution of the Vitamin E-TPGS into water. Encapsulation of paclitaxel can occur over time, and is driven by partitioning of paclitaxel into the dynamic micelles present in the aqueous matrix.

However, the ethanol/Vitamin E-TPGS formulation maintained a slight turbidity. This observation suggested that paclitaxel may not be completely soluble in this composition, or that Vitamin E-TPGS may not be completely miscible in ethanol.

To overcome or avoid the possible physical incompatibility between ethanol and Vitamin E-TPGS many of the variables were adjusted.

2.255 g of ethanol, 0.020 g of citric acid, and 0.25 g of water were combined in a 20 ml scintillation vial. The mixture was agitated slightly to dissolve the citric acid. To the ethanol and citric acid aqueous mixture, 2.542 g of Vitamin E-TPGS was added, warmed, and stirred. The Vitamin E-TPGS liquefied at 40° C. To this warmed, stirred solution, 0.053 g of paclitaxel was added with continued stirring.

The solution was fluid and appeared very clear. There was absolutely no turbidity from either undissolved taxanes or Vitamin E-TPGS.

To test the ability of this formulation to capture the taxane into micelles, 5 drops of the formulation was transferred to 5 ml of water. No turbidity was observed. Only small pieces of gelatinous material could be seen at first, which soon dissipated to yield a crystal clear solution. This formulation which was added to the water was allowed to cool to room temperature. Upon cooling, the solution was still clear. After being allowed to sit for at least 24 hours, the aqueous solution still remained clear.

### EXAMPLE 8

### Production and Characterization of Additional Parenteral Formulations

Having prepared a working formulation as shown in Example 7, additional formulations were prepared:

20% ethanol, 80% TPGS;

40% ethanol, 60% TPGS;

50% ethanol, 50% TPGS;

62.5% ethanol, 37.5% TPGS; and

75% ethanol, 25% TPGS.

To each of these formulations paclitaxel was added in the amounts of 6 mg/ml, 10 mg/ml, 20 mg/ml, and 50 mg/ml.

Citric acid was weighed into each of five 20 ml scintillation vials to which ethanol was added using a plastic transfer pipette. The mixture was agitated to dissolve the citric acid. Vitamin E-TPGS was liquefied in a 40° C. oven and then carefully poured into each scintillation vial. The vials were warmed slightly to liquefy the Vitamin E-TPGS and to accelerate dissolution into the ethanol/citric acid co-solvent mixture. See Table 7. The vials were shaken until the mixture appeared uniform. All of the solutions were allowed to cool to room temperature. Upon cooling, each of the above formulations were then distributed into 4 individual 20 ml scintillation vials, so that each vial contained approximately 4 g of the mixture. To each of these vials, 24, 40, 80, or 200 mg of paclitaxel was added. See Table 8. Each formulation was agitated until the taxane dissolved or reached an equilibrium solubility.

TABLE 7

| | Ethanol | | Citric Acid | | Vitamin E-TPGS | |
|---|---|---|---|---|---|---|
| | Desired | Actual | Desired | Actual | Desired | Actual |
| Sample ID | Amount | Amount | Amount | Amount | Amount | Amount |
| A (20%) | 3.60 g | 3.604 g | 0.036 g | 0.036 g | 14.40 g | 14.402 g |
| B (40%) | 7.20 g | 7.205 g | 0.036 g | 0.036 g | 10.80 g | 10.805 g |
| C (50%) | 9.00 g | 9.010 g | 0.036 g | 0.038 g | 9.00 g | 9.024 g |
| D (62.5%) | 11.25 g | 11.252 g | 0.036 g | 0.038 g | 6.75 g | 6.772 g |
| E (75%) | 13.50 g | 13.499 g | 0.036 g | 0.037 g | 4.50 g | 4.509 g |

TABLE 8

| | Amount of Formulation | | | |
|---|---|---|---|---|
| | Weighed into Vial | | Amount of Paclitaxel | |
| Sample | | Actual | Weighed into Vial | |
| ID | Desired Amount | Amount | Desired Amount | Actual Amount |
| A-1 | 4.00 g | 4.010 g | 0.024 g | 0.024 g |
| A-2 | 4.00 g | 4.013 g | 0.040 g | 0.041 g |
| A-3 | 4.00 g | 4.026 g | 0.080 g | 0.080 g |
| A-4 | 4.00 g | 4.027 g | 0.200 g | 0.201 g |
| B-1 | 4.00 g | 4.018 g | 0.024 g | 0.024 g |
| B-2 | 4.00 g | 4.021 g | 0.040 g | 0.041 g |
| B-3 | 4.00 g | 4.010 g | 0.080 g | 0.080 g |
| B-4 | 4.00 g | 4.004 g | 0.200 g | 0.201 g |
| C-1 | 4.00 g | 4.000 g | 0.024 g | 0.024 g |
| C-2 | 4.00 g | 4.001 g | 0.040 g | 0.040 g |
| C-3 | 4.00 g | 4.018 g | 0.080 g | 0.081 g |
| C-4 | 4.00 g | 4.006 g | 0.200 g | 0.199 g |
| D-1 | 4.00 g | 4.008 g | 0.024 g | 0.024 g |
| D-2 | 4.00 g | 4.005 g | 0.040 g | 0.042 g |
| D-3 | 4.00 g | 4.028 g | 0.080 g | 0.080 g |
| D-4 | 4.00 g | 4.003 g | 0.200 g | 0.200 g |
| E-1 | 4.00 g | 4.003 g | 0.024 g | 0.024 g |
| E-2 | 4.00 g | 4.040 g | 0.040 g | 0.041 g |
| E-3 | 4.00 g | 4.014 g | 0.080 g | 0.081 g |
| E-4 | 4.00 g | 4.009 g | 0.200 g | 0.202 g |

Solution E-4 (75% ethanol, 25% TPGS, 50 mg/ml paclitaxel) persisted in a state of turbidity, which suggested that the taxane had exceeded its solubility in this mixture. It is unlikely that the Vitamin E-TPGS was responsible for the turbidity, as it would precipitate out as small, star-like masses.

Solutions A-1 through A-4 (20% ethanol, 80% TPGS) all were viscous.

A CREMOPHOR® EL control preparation was made to provide a standard against which the Vitamin E-TPGS formulations could be measured. The same procedures used above to produce the Vitamin E-TPGS formulations were used to produce the CREMOPHOR® EL formulation, except that no warming was required, as CREMOPHOR® EL is a liquid at room temperature.

Formulations B-1 through E-3 were diluted five-fold and twenty-fold in water to monitor physical stability of the TPGS micelles in the presence of various amounts of ethanol and paclitaxel. (The A-series was not pursued further at this point, as precipitation of TPGS was apparent in 2 of the 4 preparations. Further, preparation E-4 was omitted, as the paclitaxel never completely dissolved and remained in the form of a microparticulate.) 4 g (5-fold dilution) or 9.5 g (20-fold dilution) of water was transferred to individual 20 ml scintillation vials. 1 g of the formulations was added to the vials containing the 4 g of water, and 0.5 g of the formulations was added to the vials containing 9.5 g of water. The solutions were agitated and visually inspected. The diluted solutions were then monitored for duration of physical stability.

B series (40% ethanol)—formulation solidified into a gelatinous mass which subsequently dissolved and dissipated over time; agglomerated.

C series (50% ethanol)—formulations dispersed very quickly with small fragments of gelatinous matrix visible. However, dissolution occurred very quickly.

D series (62.5% ethanol)—formulations dispersed immediately when contacting water. Dissolution occurred almost instantaneously.

E series (75% ethanol)—same as D series above.

Absolutely no turbidity was observed for any of the formulations upon introduction to the water. This observation was true despite compositional differences and the 6, 10, 20, or 50 mg/ml of paclitaxel present.

The rate at which turbidity or precipitation occurred in the solutions appears to be comparable to the behavior seen for the CREMOPHOR® EL/ethanol (5 g EtOH, 0.0205 g Citric Acid, 5 g of CREMOPHOR® EL, and 0.060 g paclitaxel) control sample when diluted into water.

All of the above solutions were visually inspected on an ongoing time basis to determine the duration of physical stability when in contact with water. See Table 9. At the 24-hour time interval, all but the solutions containing 50% ethanol:50% Vitamin E-TPGS (6 or 10 mg/ml taxanes) displayed precipitation when diluted at the 5-fold level. All of the 20-fold dilution samples retained the characteristics observed at 12 hours. The precipitate was visualized by swirling the vials in a clockwise fashion.

TABLE 9

| Sample | | | | | | | | | | | | 12 |
|---|---|---|---|---|---|---|---|---|---|---|---|---|
| ID | 1 hr | 2 hr | 3 hr | 4 hr | 5 hr | 6 hr | 7 hr | 8 hr | 9 hr | 10 hr | 11 hr | hr |
| B-1(5x) | x | x | x | x | x | x | x | x | x | x | x | x |
| B-2(5x) | x | x | x | x | x | x | x | x | x | x | x | x |
| B-3(5x) | x | x | x | x | x | x | x | x | x | x | x | x |
| B-4(5x) | x | x | ppt | | | | | | | | | |
| C-1(5x) | x | x | x | x | x | x | x | x | x | x | x | x |
| C-2(5x) | x | x | x | x | x | x | x | x | x | x | x | x |
| C-3(5x) | x | x | x | x | x | x | x | ppt | | | | |
| C-4(5x) | x | ppt | | | | | | | | | | |
| D-1(5x) | x | x | x | x | x | x | x | x | x | x | x | x |
| D-2(5x) | x | x | x | x | x | x | x | x | x | x | x | x |
| D-3(5x) | x | x | ppt | | | | | | | | | |
| D-4(5x) | x | ppt | | | | | | | | | | |
| E-1(5x) | x | x | x | x | x | x | x | x | x | x | x | x |
| E-2(5x) | x | x | x | ppt | | | | | | | | |
| E-3(5x) | ppt | | | | | | | | | | | |
| E-4(5x) | na | | | | | | | | | | | |
| Control | x | x | x | x | x | x | x | x | x | x | x | x |

Key: ppt = visual precipitate or particulate; x = clear solution.

TABLE 10

| Sample | 1 | | | | | | | | | | | 12 |
|---|---|---|---|---|---|---|---|---|---|---|---|---|
| ID | hr | 2 hr | 3 hr | 4 hr | 5 hr | 6 hr | 7 hr | 8 hr | 9 hr | 10 hr | 11 hr | hr |
| B-1(20x) | x | x | x | x | x | x | x | x | x | x | x | x |
| B-2(20x) | x | x | x | x | x | x | x | x | x | x | x | x |
| B-3(20x) | x | x | x | x | x | x | x | x | x | x | x | x |
| B-4(20x) | x | x | x | x | x | x | ppt | | | | | |
| C-1(20x) | x | x | x | x | x | x | x | x | x | x | x | x |
| C-2(20x) | x | x | x | x | x | x | x | x | x | x | x | x |
| C-3(20x) | x | x | x | x | x | x | x | x | x | x | x | x |
| C-4(20x) | x | x | x | x | x | ppt | | | | | | |
| D-1(20x) | x | x | x | x | x | x | x | x | x | x | x | x |
| D-2(20x) | x | x | x | x | x | x | x | x | x | x | x | x |
| D-3(20x) | x | x | x | x | x | x | x | x | x | x | x | ppt |
| D-4(20x) | x | x | x | ppt | | | | | | | | |
| E-1(20x) | x | x | x | x | x | x | x | x | x | x | x | x |
| E-2(20x) | x | x | x | x | x | x | x | x | x | x | x | x |
| E-3(20x) | x | x | x | x | ppt | | | | | | | |
| E-4(20x) | na | | | | | | | | | | | |
| Control | x | x | x | x | x | x | x | x | x | x | x | x |

Key: ppt = visual precipitate or particulate; x = clear solution.

### EXAMPLE 9

### Oral Feasibility PK

A preferred formulation for the oral delivery of paclitaxel was evaluated for safety in mammals using dogs. The formulation was as follows:

| | | |
|---|---|---|
| Vitamin E TPGS | Eastman | 70% |
| Dimethylisosorbide | ICI | 20% |
| paclitaxel | NBT | 10% |
| Citric acid (anhydrous) | Sigma | 2 mg/g |

and was hand filled into single 0 hard gelatin capsules to a dose of approximately 34 mg/capsule.

Six male beagles, all approximately six months old, were given paclitaxel capsules at doses from 3 to 11 milligram of drug per kilogram of body weight. Capsules were inserted into the esophagus and followed with a brief squirt of water and the animals held until swallowing was observed. Fourteen blood samples—16 hours predose through 48 hours postdose—were drawn from each animal and the plasma analyzed for paclitaxel by a sensitive and specific HPLC method. Five of the six animals had detectable levels of intact drug following dosing. The plasma level data have been reduced by noncompartmental analysis and the results are presented in Table 11.

TABLE 11
Model-independent bioavailability summary

| | Mass, | Caps | Dose, | Cₘₐₓ¹, | AUC², | | |
|---|---|---|---|---|---|---|---|
| Animal | kg | given | mg/kg | ng/mL | nghr/mL | tₘₐₓ³, hr | tₗₐₛₜ⁴, hr |
| PXF-8 | 10.4 | 1 | 3.23 | 17 | 194 | 1 | 12 |
| OEF-8 | 10.3 | 1 | 3.27 | 97 | 187 | 1 | 8 |
| QEF-8 | 10.2 | 1 | 3.30 | nd⁵ | nd | nd | nd |
| IOF-8 | 14.8 | 3 | 6.88 | 92 | 135 | 0.5 | 6 |
| FJF-8 | 13.4 | 3 | 7.66 | 108 | 202 | 1 | 8 |
| OLF-8 | 9.3 | 3 | 11.14 | 63 | 189 | 1 | 8 |

These data confirm the systemic availability of paclitaxel following oral administration of this formulation.
¹Maximum paclitaxel concentration observed in the plasma
²Calculated as the sum of trapezoids
³Time of maximum concentration observed
⁴Time of last nonzero concentration observed
⁵No drug detected

### EXAMPLE 10

### Composition of an Injection Concentrate

In all of the following examples paclitaxel is used as the taxane component. It should be noted that paclitaxel can be substituted with other taxanes.

Mixing Instructions for Examples 10.1 through 10.4:

The citric acid is dissolved in the ethanol. The desired amount of Vitamin E TPGS is warmed to approximately 40° C. with stirring. To the warmed Vitamin E TPGS, is added the solvent, ethanol containing citric acid, with stirring while maintaining a temperature of 40° C. The solution is stirred until uniform and taxanes are added slowly with continued stirring. Upon complete dissolution of the taxanes, the solution is allowed to cool to room temperature. The solution remains fluid even after equilibrating to ambient room temperature.

### EXAMPLE 10.1

| COMPONENT | QUANTITY |
|---|---|
| Ethanol | 400 mg |
| Citric Acid | 2 mg |
| Vitamin E TPGS | 600 mg |
| Paclitaxel | † |

†6, 10, 20, or 50 mg

### EXAMPLE 10.2

| COMPONENT | QUANTITY |
|---|---|
| Ethanol | 500 mg |
| Citric Acid | 2 mg |
| Vitamin E TPGS | 500 mg |
| Paclitaxel | † |

†6, 10, 20, or 50 mg

### EXAMPLE 10.3

| COMPONENT | QUANTITY |
|---|---|
| Ethanol | 625 mg |
| Citric Acid | 2 mg |
| Vitamin E TPGS | 375 mg |
| Paclitaxel | † |

†6, 10, 20, or 50 mg

### EXAMPLE 10.4

| COMPONENT | QUANTITY |
|---|---|
| Ethanol | 725 mg |
| Citric Acid | 2 mg |
| Vitamin E TPGS | 250 mg |
| Paclitaxel | † |

†6, 10, 20, or 50 mg

Mixing Instructions for Examples 10.5 through 10.8:

The desired amount of Vitamin E TPGS is warmed to approximately 40° C. with stirring. To the warmed Vitamin E TPGS, is added the solvent, ethanol, with stirring while maintaining a temperature of 40° C. The solution is stirred until uniform and taxanes are added slowly with continued stirring. Upon complete dissolution of the taxanes, the solution is allowed to cool to room temperature. The solution remains fluid even after equilibrating to ambient room temperature.

### EXAMPLE 10.5

| COMPONENT | QUANTITY |
|---|---|
| Ethanol | 400 mg |
| Vitamin E TPGS | 600 mg |
| Paclitaxel | † |

†6, 10, 20, or 50 mg

### EXAMPLE 10.6

| COMPONENT | QUANTITY |
|---|---|
| Ethanol | 500 mg |
| Vitamin E TPGS | 500 mg |
| Paclitaxel | † |

†6, 10, 20, or 50 mg

### EXAMPLE 10.7

| COMPONENT | QUANTITY |
|---|---|
| Ethanol | 625 mg |
| Vitamin E TPGS | 375 mg |
| Paclitaxel | † |

†6, 10, 20, or 50 mg

### EXAMPLE 10.8

| COMPONENT | QUANTITY |
|---|---|
| Ethanol | 725 mg |
| Vitamin E TPGS | 250 mg |
| Paclitaxel | + |

+ 6, 10, 20, or 50 mg

### EXAMPLE 11

### Composition of a Gelatin Capsule

Mixing Instructions for Examples 11.1 through 11.3:

The citric acid is dissolved in the CREMOPHOR® EL cosurfactant. The desired amount of Vitamin E TPGS is warmed to approximately 40° C. with stirring. To the warmed Vitamin E TPGS, is added the CREMOPHOR® EL/citric acid mixture with stirring while maintaining a temperature of 40° C. To the warmed mixture, is added the solvent of choice with stirring while maintaining a temperature of 40° C. The solution is stirred until uniform and taxanes are added slowly with continued stirring. Upon complete dissolution of the taxanes, the solution is allowed to cool to room temperature.

### EXAMPLE 11.1

| | |
|---|---|
| COMPONENT | QUANTITY |

| | | |
|---|---|---|
| CREMOPHOR ® EL | 100 | mg |
| Citric Acid | 2 | mg |
| Dimethylisosorbide | 250 | mg |
| Vitamin B TPGS | 550 | mg |
| Paclitaxel | 100 | mg |

### EXAMPLE 11.2

| | |
|---|---|
| COMPONENT | QUANTITY |

| | | |
|---|---|---|
| CREMOPHOR ® EL | 100 | mg |
| Citric Acid | 2 | mg |
| Dimethylisosorbide | 300 | mg |
| Vitamin E TPGS | 500 | mg |
| Paclitaxel | 100 | mg |

### EXAMPLE 11.3

| | |
|---|---|
| COMPONENT | QUANTITY |

| | | |
|---|---|---|
| CREMOPHOR ® EL | 100 | mg |
| Citric Acid | 2 | mg |
| Dimethylisosorbide | 250 | mg |
| Vitamin E TPGS | 590 | mg |
| Paclitaxel | 60 | mg |

Mixing Instructions for Examples 11.4 through 11.8:

The desired amount of Vitamin E TPGS is warmed to approximately 40° C. with stirring. To the warmed Vitamin E TPGS, is added the solvent of choice with stirring while maintaining a temperature of 40° C. If present, a thickener (e.g. PEG 4600) is added with stirring while maintaining a temperature of 40° C. The solution is stirred until uniform and taxanes are added slowly with continued stirring. Upon complete dissolution of the taxanes, the solution is allowed to cool to room temperature.

### EXAMPLE 11.4

| | |
|---|---|
| COMPONENT | QUANTITY |

| | | |
|---|---|---|
| Dimethylisosorbide | 200 | mg |
| Vitamin E TPGS | 700 | mg |
| Paclitaxel | 100 | mg |

### EXAMPLE 11.5

| | |
|---|---|
| COMPONENT | QUANTITY |

| | | |
|---|---|---|
| Dimethylisosorbide | 250 | mg |
| Vitamin E TPGS | 650 | mg |
| Paclitaxel | 100 | mg |

### EXAMPLE 11.6

| | |
|---|---|
| COMPONENT | QUANTITY |

| | | |
|---|---|---|
| Methoxy PEG 350 | 250 | mg |
| Vitamin E TPGS | 650 | mg |
| Paclitaxel | 100 | mg |

### EXAMPLE 11.7

| | |
|---|---|
| COMPONENT | QUANTITY |

| | | |
|---|---|---|
| PEG 300 | 250 | mg |
| Vitamin E TPGS | 650 | mg |
| Paclitaxel | 100 | mg |

### EXAMPLE 11.8

| | |
|---|---|
| COMPONENT | QUANTITY |

| | | |
|---|---|---|
| PEG 4600 Flake | 50 | mg |
| Dimethylisosorbide | 250 | mg |
| Vitamin E TPGS | 700 | mg |
| Paclitaxel | 100 | mg |

It should be understood that the examples and embodiments described herein are for illustrative purposes only and that various modifications or changes in light thereof will be suggested to persons skilled in the art and are to be included within the spirit and purview of this application and the scope of the appended claims.

### References

(1989) The Merck Index monograph 9040.

(1991) U.S. National Cancer Institute's Clinical Brochure for Taxol.

(1992) Second National Cancer Institute Workshop on Taxol and Taxus held in Alexandria, Va. U.S.A.

U.S. Pat. No. 4,942,184, Issued Jul. 17, 1990.

U.S. Pat. No. 4,960,790, Issued Oct. 2, 1990.

U.S. Pat. No. 5,733,888, Issued Mar. 31, 1998.

What is claimed is:

1. A composition useful for treating a taxane-responsive disease condition, comprising a taxane and at least one compound selected from the group consisting of Vitamin E-TPGS, dimethylisosorbide (DMI), methoxy PEG 350, citric acid, PEG 300, and PEG 4600.

2. The composition of claim 1 comprising taxane and Vitamin E-TPGS.

3. The composition of claim 1 comprising about 65% to about 70% Vitamin E-TPGS, about 20% to about 25% DMI, and about 5% to about 10% taxane.

4. The composition of claim 3 further comprising citric acid.

5. The composition of claim 1 comprising ethanol, Vitamin E-TPGS, and taxane.

6. The composition of claim 5 comprising about 25% to about 75% Vitamin E-TPGS, about 25% to about 75% ethanol, and about 0.6% to about 5% taxane.

7. The composition of claim 6 further comprising about 0.2% citric acid.

8. The composition of claim 7 further comprising about 10% polyethoxylated castor oil.

9. The composition of claim 1 comprising polyethoxylated castor oil, citric acid, dimethylisosorbide, Vitamin E-TPGS, and taxane.

10. The composition of claim 9 comprising about 10% polyethoxylated castor oil, about 25% to about 30% dimethylisosorbide, about 50% to about 60% Vitamin E-TPGS, about 0.2% citric acid, and about 5% to about 10% taxane.

11. The composition of claim 1 wherein the taxane is paclitaxel or derivatives, analogues, or prodrugs thereof.

12. The composition of claim 1 comprising methoxy PEG 350, Vitamin E-TPGS, and taxane.

13. The composition of claim 12 comprising about 25% methoxy PEG 350, about 65% Vitamin E-TPGS, and about 10% taxane.

14. The composition of claim 1 comprising PEG 300, Vitamin E-TPGS, and taxane.

15. The composition of claim 14 comprising about 25% PEG 300, about 65% Vitamin E-TPGS, and about 10% taxane.

16. The composition of claim 1 comprising PEG 4600, dimethylisosorbide, Vitamin E-TPGS, and taxane.

17. The composition of claim 16 comprising about 5% PEG 4600, about 25% dimethylisosorbide, about 70% Vitamin E-TPGS, and about 10% taxane.

18. A method of treating a taxane-responsive disease condition comprising the steps of:
obtaining a composition comprising a taxane and at least one compound selected from the group consisting of Vitamin E-TPGS, dimethylisosorbide, polyethoxylated castor oil, ethanol, methoxy PEG 350, citric acid, PEG 300, and PEG 4600; and
administering said composition to a mammal having a taxane-responsive disease condition.

19. The method of claim 18 wherein said composition comprises about 65% to about 70% Vitamin E-TPGS, about 20% to about 25% dimethylisosorbide, about 5% to about 10% taxane.

20. The method of claim 19 further comprising citric acid.

21. The method of claim 18 wherein said composition comprises about 25% to about 75% Vitamin E-TPGS, about 25% to about 75% ethanol, and about 0.6% to about 5% taxane; and wherein administering comprises intravenous or parenteral administration.

22. The method of claim 21 wherein the composition further comprises about 0.2% citric acid.

23. The method of claim 18 wherein said composition comprises about 10% polyethoxylated castor oil, about 25% to about 30% dimethylisosorbide, about 50% to about 60% Vitamin E-TPGS, and about 5% to about 10% taxane; and wherein administering said composition comprises oral administration.

24. The method of claim 18 wherein said composition comprises about 25% PEG 300, about 65% Vitamin E-TPGS, and about 10% taxane.

25. The method of claim 18 wherein said composition comprises about 5% PEG 4600, about 25% dimethylisosorbide, about 70% Vitamin E-TPGS, and about 10% taxane.

26. The method of claim 18 wherein said taxane-responsive disease condition is selected from the group consisting of ovarian cancer, prostate cancer, breast cancer, malignant lymphoma, lung cancer, melanoma, Kaposi's sarcoma, polycystic kidney disease, Alzheimer's disease, malaria, and rheumatoid arthritis.
